(19) United States
(12) Patent Application Publication
(10) Pub. No.: US 2014/0102444 A1
(43) Pub. Date: Apr. 17, 2014

(54) VENTILATION CONDUIT FOR A VENTILATION SYSTEM FOR THE DELIVERY OF AIR, VENTILATION SYSTEM FOR THE DELIVERY OF AIR AND METHOD FOR CLEANING A VENTILATION CONDUIT

(71) Applicant: Robert Bosch GmbH, Stuttgart (DE)

(72) Inventor: Victoria Perez-Solorzano Borragan, Stuttgart (DE)

(73) Assignee: Robert Bosch GmbH, Stuttgart (DE)

(21) Appl. No.: 14/051,611

(22) Filed: Oct. 11, 2013

(30) Foreign Application Priority Data
Oct. 15, 2012 (DE) 10 2012 218 734.0

(51) Int. Cl. A61B 19/00; A61M 16/08

(52) U.S. Cl. CPC A61B 19/34; A61M 16/0875
USPC 128/202.13

(57) ABSTRACT

A ventilation conduit for a ventilation system for the delivery of air includes an outer electrode and in inner electrode. The outer electrode is formed as a wall body of the ventilation conduit. The inner electrode is configured to be arranged in an interior space of the ventilation conduit that is enclosed by the outer electrode. Applying an electric voltage between the inner electrode and the outer electrode allows a gas discharge to be brought about to generate a plasma for cleaning the ventilation conduit in an installed state.

This application claims priority under 35 U.S.C. §119 to patent application no. DE 10 2012 218 734.0, filed on Oct. 15, 2012, the disclosure of which is incorporated herein by reference in its entirety.

### BACKGROUND

The present disclosure relates to a ventilation conduit for a ventilation system for the delivery of air, to a ventilation system for the delivery of air, to a method for cleaning a ventilation conduit of a ventilation system for the delivery of air, to a corresponding control unit and to a corresponding computer program product.

Conduits or tubes of ventilation systems are usually cleaned, in particular mechanically by means of robots. In addition to procurement costs arising due to use of such robots, which may be caused by pipe systems that are large enough for the use of robots, corresponding regular servicings of ventilation systems must also be provided. VDI Standard 6022, for example, obliges operators of ventilation systems to carry out regular servicing and cleaning to optimize health protection and system reliability. Mechanical cleaning, for example by means of hot and cold water as well as by processes that use steam, high pressure and low pressure, can free a ventilation duct of dust, dead insects, animals and the like, but in particular there is usually no disinfection, so that further detrimental effects to human health may occur. VDI Standard 6038, for example, includes an assessment of the contents of inhaled air, in order to assess the quality of inhaled air.

DE 600 23 757 T2 discloses a plasma disinfection system with a reaction chamber for receiving an article to be sterilized.

### SUMMARY

Against this background, the approach proposed here presents a ventilation conduit for a ventilation system for the delivery of air, a ventilation system for the delivery of air, a method for cleaning a ventilation conduit of a ventilation system for the delivery of air, also a control unit that performs this method and finally a corresponding computer program product according to the description below. Advantageous configurations are provided by the description that follows.

According to embodiments of the present disclosure, an integrated in-situ cleaning or in-situ disinfection for ventilation conduits or tubes installed in ventilation systems can be made possible in particular by a special structural configuration of a ventilation conduit. Here, ventilation ducts are, for example, not only used for the transporting of air but also configured such that, by the application of an electric voltage, a plasma can be produced in the interior space for the purpose of cleaning. In this way, a disinfection of the conduit can also be realized. For the cleaning, a gas discharge with plasma generation can be made possible in particular by the special construction of the ventilation conduit and by purpose-built ventilation system components.

Advantageously, embodiments of the present disclosure make cleaning of a ventilation conduit possible in the installed state of the same, in particular an in-situ cleaning of ventilation conduits in the sense of disinfection. Consequently, ventilation conduits can be cleaned in particular in the position in which they are used and in the state in which they are used, without removing them. At the same time, it is also possible, for example, to achieve the killing of bacteria, viruses and mold fungi to optimize the protection of health and system reliability. Even complicated conduit geometries can be cleaned and, for example, automatic cleaning at definable intervals is possible.

The approach proposed here provides a ventilation conduit for a ventilation system for the delivery of air, the ventilation conduit having the following features:
an outer electrode, which is formed as a wall body of the ventilation conduit; and
an inner electrode, which can be arranged or is arranged in an interior space of the ventilation conduit that is enclosed by the outer electrode, it being possible by applying an electric voltage between the inner electrode and the outer electrode to bring about a gas discharge, in order to generate a plasma for cleaning the ventilation conduit in an installed state.

The ventilation conduit may comprise a tube, a pipe, a pipeline, a duct or the like for the delivery of air. The ventilation conduit may have a round, oval or angular cross-sectional profile. The ventilation conduit may comprise the wall body that encloses the interior space of the ventilation conduit. In the interior space of the ventilation conduit, it is possible that a stream of gas and a stream of air can be conducted. The wall body of the ventilation conduit may be formed from a metal. The wall body of the ventilation conduit may represent the outer electrode. The outer electrode may comprise the wall body of the ventilation conduit. The outer electrode may comprise an electrically conductive material. The inner electrode may comprise an electrically conductive material, in particular a metal. It may be that the inner electrode can be arranged or is arranged in the interior space of the ventilation conduit at a distance from the outer electrode or the wall body of the ventilation conduit. In this way, an intermediate space is provided between the inner electrode and the outer electrode in the interior space of the ventilation conduit. The electric voltage that can be applied between the inner electrode and the outer electrode may be a high voltage. A high voltage may be understood as meaning a voltage that has a voltage value of over 1000 V. With the applied electric voltage, the inner electrode may be at a first electric potential and the outer electrode may be at a second electric potential. The electric potential of the outer electrode may be a ground potential. In the event that, for functional reasons, the voltage should be on the outer electrode, an insulation/sheathing of the pipe is necessary in order to ensure corresponding shock protection.

In particular, a grounding device may be provided for grounding the outer electrode. In this way, the outer electrode or the wall body can be grounded or is grounded and, as a result, is safe and of no concern for persons, for example users.

The inner electrode may also be enclosed by a dielectric. In particular, the inner electrode may comprise a metal wire sheathed with a dielectric. Such an embodiment offers the advantage that a silent electrical discharge or dielectric barrier discharge can be generated as a gas discharge, it being possible for a volume of gas or volume of air between the dielectrically enclosed inner electrode and the outer electrode to be advantageously ionized or transformed into a plasma state by means of a suitable electric voltage or field strength.

The present disclosure also provides a ventilation system for the delivery of air that has the following features:
at least one aforementioned ventilation conduit; and
a high-voltage source, which can be connected or is connected electrically to the inner electrode of the ventilation conduit, in order to apply the electric voltage between the inner electrode and the outer electrode.

The ventilation system may be advantageously used in conjunction with the aforementioned ventilation conduit, in order to achieve cleaning of the ventilation conduit. Here, the inner electrode that can be integrated or is integrated in the interior space of the ventilation conduit can be supplied with high voltage, in order to bring about the gas discharge in the interior space.

Furthermore, a gas supplying device may be provided, configured to provide at least one gas for the plasma generation in the interior space of the ventilation conduit. The gas supplying device may be configured to introduce a gas or a gas mixture of multiple gases into the interior space of the ventilation conduit. The at least one gas may in particular be oxygen. The gas supplying device may be configured to introduce the at least one gas in a definable ratio to the air that is delivered. Consequently, depending on the application of the ventilation system, not only can air be delivered but, in addition or as an alternative to air, it is also possible to use gases provided by means of the gas supplying device, such as oxygen, for the plasma generation. Such an embodiment offers the advantage that a further improved cleaning effect can be achieved, in particular on account of the formation of ozone in the case of plasma generation after the introduction of oxygen into the interior space of the ventilation conduit. In this way, particularly advantageous cleaning of the ventilation conduit is made possible by means of the ventilation system, in particular by means of the high-voltage source and the gas supplying device of the same. The supplying of the gas may also take place through the inner pipe. The inner pipe should then be correspondingly “spot-drilled”, in order to form the gas outlets.

Moreover, a gas sensor device may be provided for sensing substances in the interior space of the ventilation conduit. In this case, at least one sensing element of the gas sensor device may be arranged in the interior space of the ventilation conduit. By means of the gas sensor device, pollutants in particular can be detected when the ventilation system is improperly used. This allows reliable operation of the ventilation system to be achieved. Ozone detection is also possible, for example, before opening the system prior to repairs.

The approach presented here also provides a method for cleaning a ventilation conduit of an aforementioned ventilation system for the delivery of air, the method having the following step:
applying an electric voltage between the inner electrode and the outer electrode of the ventilation conduit, in order when there is a gas discharge brought about as a result of the voltage to generate a plasma for cleaning the ventilation conduit in an installed state.

The method may be advantageously performed in conjunction with the aforementioned ventilation system with the aforementioned ventilation conduit, in order to achieve cleaning of the ventilation conduit. In this way, the aforementioned ventilation conduit or the aforementioned ventilation system can be advantageously used in conjunction with the method.

The approach presented here also provides a control unit that is configured to carry out or implement the step of the aforementioned method in corresponding devices. This variant of an embodiment of the disclosure in the form of a control unit also allows the object on which the disclosure is based to be achieved quickly and efficiently.

A control unit may be understood in the present case as meaning an electrical unit that processes cleaning signals, for example sensor signals and/or user input signals, and outputs on the basis thereof control signals, in particular activation signals, to the high-voltage source of the ventilation system. The control unit may be configured to bring about automatic, plannable cleaning or disinfection of ventilation conduits by means of repeated performance of the method, for example at predefined time intervals. The control unit may have an interface, which may be formed by hardware and/or software. In a case in which they are formed by hardware, the interfaces may, for example, be part of a so-called system ASIC, which comprises a wide variety of functions of the control unit. It is also possible, however, that the interfaces are dedicated integrated circuits, or at least partially consist of discrete components. In a case in which they are formed by software, the interfaces may be software modules that are present, for example, on a microcontroller along with other software modules. For larger ventilation systems, it is favorable for the disinfection to take place in phases, so that the inner electrode may be made such that it can be switched phase by phase, and it can be switched or can be operated by the control unit and/or the corresponding software.

Also of advantage is a computer program product with program code, which may be stored on a machine-readable carrier such as a semiconductor memory, a hard disk storage device or an optical storage device and, for carrying out the method, is used on the basis of one of the embodiments described above when the program product is performed on a computer or an apparatus.

### BRIEF DESCRIPTION OF THE DRAWINGS

The approach presented here is explained in more detail below by way of example on the basis of the appended drawings, in which:

FIG. 1 shows a schematic representation of a ventilation system according to an exemplary embodiment of the present disclosure; and

FIG. 2 shows a flow diagram of a method according to an exemplary embodiment of the present disclosure.

### DETAILED DESCRIPTION

In the description that follows of preferred exemplary embodiments of the present disclosure, the same or similar reference numerals are used for the elements that are represented in the various figures and act similarly, without a repeated description of these elements.

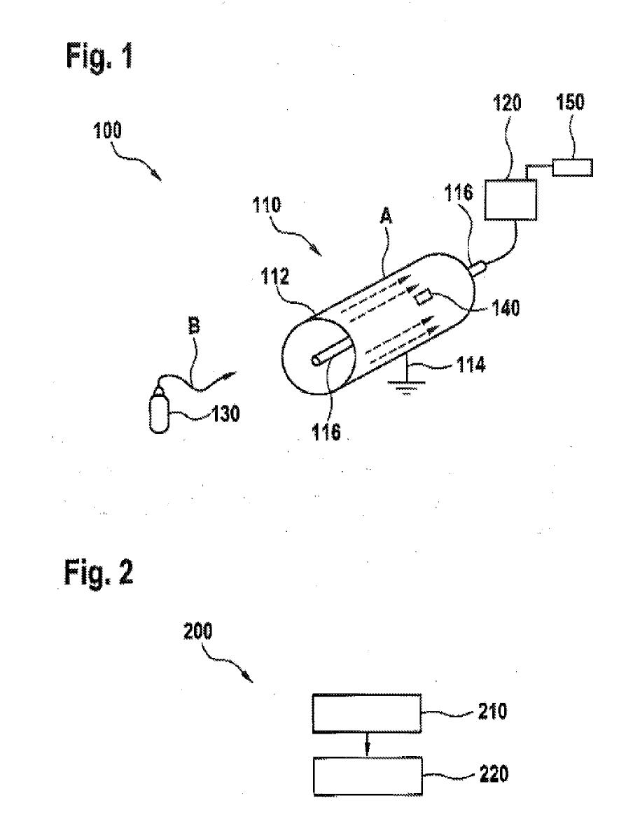

FIG. 1 shows a schematic representation of a ventilation system 100 for the delivery of air, according to an exemplary embodiment of the present disclosure. The ventilation system 100 has, merely by way of example, a ventilation conduit 110, which has an outer electrode 112, a grounding device 114 and an inner electrode 116. By means of four arrows, by way of example, a gas flow direction A, in particular an air flow direction, in the ventilation conduit 110 is symbolically represented. The ventilation system 100 also has a high-voltage source 120, a gas supplying device 130, by means of which at least one gas can be fed into the ventilation conduit 110 along a gas feeding direction B, a gas sensor device 140 and a control unit 150. In FIG. 1, a portion of the ventilation conduit 110 is shown merely by way of example.

The ventilation conduit 110 has the outer electrode 112, which is formed as a wall element of the ventilation conduit 110. According to the exemplary embodiment of the present disclosure that is represented in FIG. 1, the wall element has a round cross-sectional profile. The outer electrode 112 is grounded by means of the grounding device 114, which is electrically connected to the outer electrode 112. The outer electrode 112 or the wall element surrounds or encloses an interior space of the ventilation conduit 110. Arranged in the interior space is the inner electrode 116. The inner electrode 116 extends along the center axis or axis of longitudinal extent of the outer electrode 112 or of the wall element of the ventilation conduit 110. The inner electrode 116 is arranged at a distance from the outer electrode 112. The inner electrode is, for example, a metal wire, which optionally can be enclosed or is enclosed by a dielectric. The ventilation conduit 110 is configured to allow the flow of, or to deliver, air and possibly at least one gas from the gas supplying device 130 along the gas flow direction A.

The high-voltage source 120 is electrically connected to the inner electrode 116 of the ventilation conduit 110. The high-voltage source 120 is configured to generate an electric voltage between the inner electrode 116 and the outer electrode 112 of the ventilation conduit 110. Applying the electric voltage between the inner electrode 116 and the outer electrode 112 allows a gas discharge to be brought about, in order to generate a plasma for cleaning the ventilation conduit 110. The electric voltage is, for example, an alternating voltage, by means of which a suitable field strength for the gas discharge is brought about. The cleaning takes place here in particular in an installed state of the ventilation conduit 110.

The gas supplying device 130 is arranged and configured to provide at least one gas for the plasma generation in the interior space of the ventilation conduit 110. The gas supplying device 130 is configured to introduce a gas or a gas mixture of multiple gases into the interior space of the ventilation conduit 110 along the gas feeding direction B. The at least one gas is in particular oxygen.

The gas sensor device 140 is configured to sense gases or substances in the interior space of the ventilation conduit 110. In this case, at least one sensing element of the gas sensor device 140 is arranged in the interior space of the ventilation conduit 110. By means of the gas sensor device 140, pollutants in particular can be detected when the ventilation system 100 is improperly used.

The control unit 150 is electrically connected to the high-voltage source 120. The control unit 150 is, for example, a suitable so-called ECU (Electronic Control Unit). By means of the control unit 150, the high-voltage source 120 can be activated, in order to apply the electric voltage between the inner electrode 116 and the outer electrode 112, to generate the gas discharge and consequently the plasma for cleaning the ventilation conduit 110. Here, the control unit 150 is also configured, for example, to bring about automatic cleaning or disinfection of the ventilation conduit 110, which optionally can also be performed in a plannable and/or regular manner.

FIG. 2 shows a flow diagram of a method 200 according to an exemplary embodiment of the present disclosure. The method 200 is intended for the cleaning of a ventilation conduit of a ventilation system for the delivery of air, such as of the ventilation conduit of the ventilation system from FIG. 1. In this case, the ventilation system has at least one ventilation conduit, which has an outer electrode, which is formed as a wall body of the ventilation conduit, and an inner electrode, which can be arranged or is arranged in an interior space of the ventilation conduit that is enclosed by the outer electrode. By applying an electric voltage between the inner electrode and the outer electrode, a gas discharge can be brought about, in order to generate a plasma for cleaning the ventilation conduit in an installed state. Furthermore, the ventilation system has a high-voltage source, which can be connected or is connected electrically to the inner electrode of the ventilation conduit, in order to apply the electric voltage between the inner electrode and the outer electrode.

The method 200 has a step of reading in 210 a cleaning signal, which indicates a cleaning to be carried out of the at least one ventilation conduit. The cleaning signal is, for example, a sensor signal and/or a user input signal. The method 200 also has a step of applying 220 an electric voltage between the inner electrode and the outer electrode of the ventilation conduit, in order when there is a gas discharge brought about as a result of the voltage to generate a plasma for cleaning the ventilation conduit in an installed state. The step of applying 220 is performed by way of example in response to the cleaning signal that is read in.

In this case, the method 200 can be advantageously perfomed in conjunction with the ventilation system from FIG. 1, in particular the ventilation conduit, the high-voltage source and the control unit of the ventilation system from FIG. 1.

If an exemplary embodiment comprises an “and/or” conjunction between a first feature and a second feature, this should be read as meaning that, according to one embodiment, the exemplary embodiment comprises both the first feature and the second feature, and according to a further embodiment, the exemplary embodiment comprises either only the first feature or only the second feature.

Furthermore, method steps according to the disclosure may be performed repeatedly.

What is claimed is:

1. A ventilation conduit for a ventilation system for the delivery of air, the ventilation conduit comprising:
an outer electrode formed as a wall body of the ventilation conduit; and
an inner electrode configured to be arranged in an interior space of the ventilation conduit that is enclosed by the outer electrode,
wherein applying an electric voltage between the inner electrode and the outer electrode enables a gas discharge to generate a plasma for cleaning the ventilation conduit in an installed state.

2. The ventilation conduit according to claim 1, further comprising a grounding device configured to ground the outer electrode.

3. The ventilation conduit according to claim 1, wherein the inner electrode is enclosed by a dielectric.

4. A ventilation system for the delivery of air, the ventilation system comprising:
at least one ventilation conduit, including:
an outer electrode formed as a wall body of the at least one ventilation conduit; and
an inner electrode configured to be arranged in an interior space of the at least one ventilation conduit that is enclosed by the outer electrode,
wherein applying an electric voltage between the inner electrode and the outer electrode enables a gas discharge to generate a plasma for cleaning the at least one ventilation conduit in an installed state; and
a high-voltage source configured to be connected electrically to the inner electrode of the at least one ventilation conduit to apply the electric voltage between the inner electrode and the outer electrode.

5. The ventilation system according to claim 4, further comprising a gas supplying device configured to provide at least one gas for plasma generation in the interior space of the at least one ventilation conduit.

6. The ventilation system according to claim 4, further comprising a gas sensor device configured to sense substances in the interior space of the at least one ventilation conduit.

7. A method for cleaning a ventilation conduit of a ventilation system for the delivery of air, the method comprising:
applying an electric voltage between an inner electrode and an outer electrode of the ventilation conduit; and
generating plasma for cleaning the ventilation conduit in an installed state when there is a gas discharge brought about as a result of the voltage.

8. The method according to claim 7, wherein a control unit is configured to apply the electric voltage.

9. The method according to claim 7, wherein a computer program product with program code is configured to apply the electric voltage when the program product is performed on a control unit.
